(19) United States
(12) Patent Application Publication
(10) Pub. No.: US 2026/0224491 A1
(43) Pub. Date: Aug. 6, 2026

(54) MICROSPHERES COMPRISING HIGH-DOSE VARENICLINE, METHOD FOR PREPARING SAME, AND PHARMACEUTICAL COMPOSITION COMPRISING SAME

(71) Applicant: AULBIO CO., LTD., Seongnam-si, Gyeonggi-do (KR)

(72) Inventors: Cherng Ju KIM, Beaumont, CA (US); Tae Kun AN, Yongin-si (KR); A Ram KIM, Seoul (KR); Hyeon Ho SHIN, Suwon-si (KR); Jin PARK, Seongnam-si (KR); Yu Ri PARK, Suwon-si (KR); Gi Sun LEE, Suwon-si (KR)

(73) Assignee: AULBIO CO., LTD., Seongnam-si, Gyeonggi-do (KR)

(21) Appl. No.: 19/148,897

(22) PCT Filed: Jan. 17, 2024
(86) PCT No.: PCT/KR2024/000832

§ 371 Date: Jul. 17, 2025

(30) Foreign Application Priority Data
Jan. 18, 2023 (KR) 10-2023-0007612

(51) Int. Cl. A61K 9/16; A61K 31/499

(52) U.S. Cl. CPC A61K 9/1647; A61K 9/1682; A61K 31/499

(57) ABSTRACT

A microsphere contains varenicline hemipamoate and a biocompatible polymer. A method of preparing the microsphere, and a pharmaceutical composition containing the microsphere are disclosed. The microsphere containing varenicline encapsulates a high concentration of varenicline, and exhibits a stable drug release rate over a long period of time and an appropriate initial release rate, and thus can maintain varenicline at an effective concentration in the blood for a certain period of time without side effects.

### TECHNICAL FIELD

The present invention relates to a microsphere comprising high-dose varenicline, a method of preparing the same, and a pharmaceutical composition comprising the same.

### BACKGROUND ART

Varenicline is a compound whose chemical name is 7,8,9,10-tetrahydro-6,10-methano-6H-pyrazino[2,3-h] [3]benzazepine and has a structure represented by the following Formula 1.

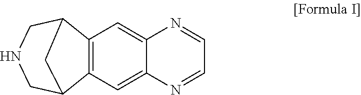 [SMILES: c1cnc2cc3c(cc2n1)C1CNCC3C1]

Varenicline, represented by the above Formula 1, is a drug useful for improving symptoms caused by cholinergic receptor activation disorder by binding to the neuronal nicotinic acetylcholine specific receptor site, and is useful for the treatment of inflammatory bowel disease, irritable bowel syndrome, spastic dystonia, chronic pain, acute pain, nontropical sprue, cystitis, vasoconstriction, anxiety disorders, panic disorder, depression, bipolar disorder, autism, sleep disorders, jet lag syndrome, amyotrophic lateral sclerosis (ALS), cognitive dysfunction, drug/toxicity-induced cognitive impairment, disease-induced cognitive impairment, hypertension, bulimia, anorexia, obesity, cardiac arrhythmias, gastric hypersecretion, ulcers, pheochromocytoma, progressive supranuclear palsy, chemical dependence and addiction (e.g., dependence or addiction to nicotine or tobacco products, alcohol, benzodiazepines, barbiturates, opioids, or cocaine), headache, migraine, stroke, traumatic brain injury (TBI), obsessive-compulsive disorder (OCD), psychosis, Huntington's chorea, tardive dyskinesia, hyperkinesia, dyslexia, schizophrenia, multi-infarct dementia, age-related cognitive decline, epilepsy including absence seizures (absence epilepsy), attention deficit hyperactivity disorder (ADHD), Tourette syndrome, and especially treatment of nicotine dependence, addiction and withdrawal in smoking cessation therapy.

Currently, varenicline tartrate salt is the main ingredient in smoking cessation treatment drugs sold worldwide under the trade names Champix® or Chantix®. Champix® is a partial agonist for α4β2 neuronal nicotinic receptors, and is a smoking cessation treatment adjunct drug that binds to acetylcholine receptors in the brain instead of nicotine to relieve smoking cravings and withdrawal symptoms.

The Champix® is known to have common side effects such as nausea, insomnia, constipation, abdominal distention, and vomiting. In particular, nausea is temporary, but some patients have difficulty continuing to take the medication, and problems such as persistent nausea occur. It has been confirmed through clinical trials that these symptoms increase in a dose-dependent manner.

Therefore, to solve these problems, the drug is administered through dose titration. First, 0.5 mg of varenicline is administered once a day for 3 days, and then 0.5 mg is administered twice a day from the 4th to the 7th day. After that, 1.0 mg of varenicline is administered twice a day until the 12th week to maintain the effective blood concentration and alleviate withdrawal symptoms.

However, this complex dosing method has the disadvantage of significantly reducing medication compliance. Therefore, development is being made for sustained-release preparations that contain varenicline as the main ingredient and can improve the convenience of taking the medication, and the microsphere system (microparticle system) is being actively studied as such sustained-release preparations.

However, in microsphere systems (microparticle systems), a high initial burst occurs in many cases. Since this initial burst can cause adverse effects including toxic reactions, it is required in the development of microparticle systems to eliminate or at least minimize this initial burst.

On the other hand, when reducing the initial release by controlling the formulation and manufacturing process, the overall release profile usually changes as well. Therefore, it is very difficult to obtain a drug release profile that is constant and continuous over a long period of time while reducing the initial release.

### PRIOR DOCUMENT

### Patent Document

(Patent Document 1) Korean Patent Registration No. 10-0551184

### DISCLOSURE

### Technical Problem

The inventors of the present invention have conducted repeated studies to solve the above-mentioned problems of the prior art and have developed a microsphere in which varenicline is encapsulated at a high concentration and the varenicline is constantly and continuously released for a long period of time.

Therefore, the present invention aims to provide a microsphere in which varenicline is encapsulated at a high concentration, is continuously released at a constant rate for a long period of time, and exhibits an appropriate initial burst of release, a method of preparing the same, and a pharmaceutical composition comprising the same.

### Technical Solution

To solve the above problem, the present invention provides a microsphere comprising varenicline hemipamoate and a biocompatible polymer.

In addition, the present invention provides a method of preparing a microsphere, comprising the following steps of:
(a) dispersing varenicline hemipamoate and a biocompatible polymer in one or more solvents to prepare a dispersed phase by;
(b) adding the produced dispersed phase to a continuous phase and stirring to form a microsphere; and
(c) removing the solvent.

In addition, the present invention provides a pharmaceutical composition for the prevention or treatment of diseases caused by cholinergic receptor activation disorder comprising the microsphere of the present invention.

### Advantageous Effects

The microsphere of the present invention provides an effect of significantly improving the encapsulation rate of varenicline by encapsulating varenicline hemipamoate in a biocompatible polymer.

In addition, the microsphere of the present invention provides the effect of continuously and steadily releasing varenicline for a long period of time without rapid initial release.

In addition, the method of preparing a microsphere including varenicline hemipamoate of the present invention provides an effect of efficiently preparing the microsphere.

In addition, the pharmaceutical composition of the present invention provides an excellent effect in the prevention and treatment of diseases caused by cholinergic receptor activation disorder by including the microsphere.

### DESCRIPTION OF DRAWINGS

FIG. 1 is a photograph illustrating the results of observing the morphology of microspheres prepared in Example 2 and Comparative Example 2 and Comparative Example 3 of the present invention using a scanning electron microscopy (SEM),

FIG. 2 is a graph illustrating the experimental results evaluating the release rate of microspheres prepared in Example 2 and Comparative Example 2, which were encapsulated with varenicline,

FIG. 3 is a graph illustrating the NMR analysis results of varenicline hemipamoate crystals prepared in Example 1,

FIG. 4 is a graph illustrating the XRD analysis results of varenicline hemipamoate crystals prepared in Example 1,

FIG. 5 is a graph illustrating the results of analyzing varenicline hemipamoate crystals prepared in Example 1 using a thermogravimetric analyzer (TGA/DSC), and

FIG. 6 is a graph illustrating the NMR analysis results of varenicline pamoate crystals prepared in Comparative Example 1.

### BEST MODE

Hereinafter, the present invention will be described in more detail.

All technical terms used in the present invention, unless otherwise defined, are used with the same meaning as generally understood by those skilled in the art in the relevant field of the present invention. In addition, those similar or equivalent to those described as preferred methods or samples in the present invention are also included in the scope of the present invention. The contents of all publications mentioned as references in this specification are incorporated herein by reference in their entirety.

The present invention relates to a microsphere comprising varenicline hemipamoate and a biocompatible polymer.

The varenicline is a compound whose chemical name is 7,8,9,10-tetrahydro-6,10-methano-6H-pyrazino[2,3-h] [3]benzazepine and has a structure represented by the following Formula 1.

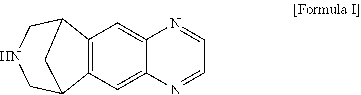 [SMILES: c1cnc2cc3c(cc2n1)C1CNCC3C1]

The varenicline is a drug that binds to the neuronal nicotinic acetylcholine specific receptor site and is useful for improving symptoms caused by cholinergic receptor activity disorder, and is known to be useful for treating inflammatory bowel disease, irritable bowel syndrome, spastic dystonia, chronic pain, acute pain, nontropical sprue, cystitis, vasoconstriction, anxiety disorders, panic disorder, depression, bipolar disorder, autism, sleep disorders, jet lag syndrome, amyotrophic lateral sclerosis (ALS), cognitive dysfunction, drug/toxicity-induced cognitive impairment, disease-induced cognitive impairment, hypertension, bulimia, anorexia, obesity, cardiac arrhythmias, gastric hypersecretion, ulcers, pheochromocytoma, progressive supranuclear palsy, chemical dependence and addiction (e.g., dependence or addiction to nicotine or tobacco products, alcohol, benzodiazepines, barbiturates, opioids, or cocaine), headache, migraine, stroke, traumatic brain injury (TBI), obsessive-compulsive disorder (OCD), psychosis, Huntington's chorea, tardive dyskinesia, hyperkinesia, dyslexia, schizophrenia, multi-infarct dementia, age-related cognitive decline, epilepsy including absence seizures (absence epilepsy), attention deficit hyperactivity disorder (ADHD), and Tourette syndrome. It is particularly useful in the treatment of nicotine dependence, addiction and withdrawal, including use in smoking cessation therapy.

The varenicline hemipamoate of the present invention has the following chemical structure:

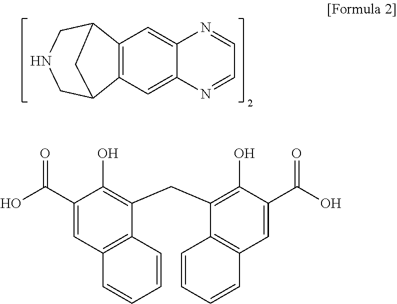

The varenicline hemipamoate is a sparingly soluble salt with low solubility, and is therefore advantageous for encapsulation in microspheres. In particular, since it exhibits an appropriate initial burst when encapsulated in microspheres, it has the advantage of being free from side effects, including toxic reactions due to excessive initial burst. In addition, it can be used very usefully because it enables obtaining a drug release profile that is constantly continued for a long period of time along with the appropriate initial release.

The content range of varenicline hemipamoate included in the microsphere may have a lower limit of 2 wt % or more, 4 wt % or more, 6 wt % or more, 8 wt % or more, 10 wt % or more, or 14 wt % or more, and an upper limit of 50 wt % or less, 45 wt % or less, 40 wt % or less, 35 wt % or less, or 30 wt % or less, based on the total weight of the microsphere. The content range of varenicline hemipamoate may be set by a combination of any of the lower limit values and any of the upper limit values.

Specifically, the content range of the varenicline hemipamoate may be 2 to 50 wt %, 4 to 45 wt %, 6 to 40 wt %, 8 to 35 wt %, 10 to 30 wt %, or 14 to 30 wt %.

If the content of varenicline hemipamoate contained in the microsphere exceeds 50 wt %, the initial release amount of varenicline in the body environment may be too high, which may cause a problem in which the blood concentration of the drug rapidly increases. If the content of varenicline hemipamoate contained in the microsphere is less than 2 wt %, the ratio of biocompatible polymer may be relatively high, making it difficult to release varenicline.

In the present invention, the content of varenicline contained in the microsphere may be 5 wt % or more, 6 wt % or more, 7 wt % or more, 8 wt % or more, 9 wt % or more, or 9.5 wt % or more, based on varenicline free base.

The varenicline hemipamoate can be prepared by a conventional technique, for example, converting free varenicline into an acid addition salt by adding pamoic acid. For example, it can be prepared by dissolving varenicline and pamoic acid in a solvent, reacting them, and then crystallizing varenicline hemipamoate.

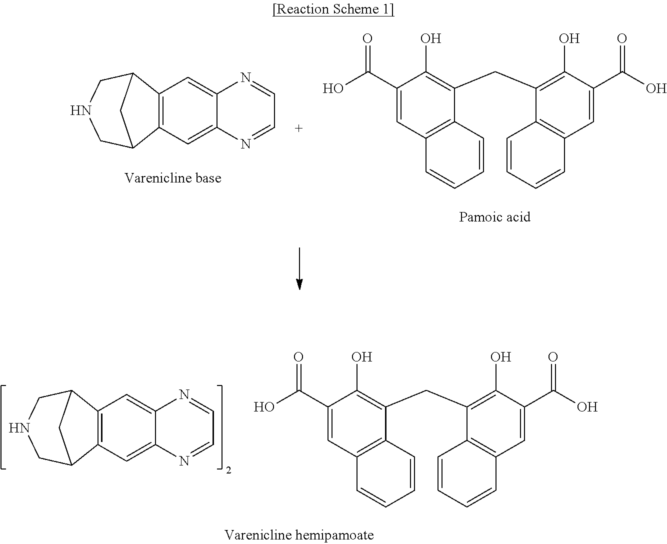

In the above, the dissolution can be carried out, for example, by heating a mixture of varenicline, pamoic acid, and a solvent to a temperature of 30 to 90° C., preferably 50 to 80° C.

In the above, the crystallization can be performed, for example, by a method of precipitating varenicline hemipamoate while cooling the varenicline and pamoic acid solution.

The solvent in the above is not particularly limited, but for example, a mixed solvent of water and dimethyl sulfoxide can be used, and ultrapure water can be preferably used as the water.

In the present invention, microsphere means a microsphere prepared using a biocompatible polymer in which the varenicline hemipamoate is encapsulated, and is simply referred to as a varenicline-containing microsphere, a varenicline microsphere, or a microsphere. If varenicline hemipamoate is encapsulated in a microsphere prepared using a biocompatible polymer, it is included in the scope of the present invention regardless of the type of biocompatible polymer used.

In the present invention, the “biocompatible polymer” refers to a polymer that has secured in vivo safety by not causing high cytotoxicity and inflammatory responses when administered in vivo, and is also referred to simply as a polymer in this specification.

In the present invention, the biocompatible polymer can be selected based on its intrinsic viscosity. A suitable intrinsic viscosity is 0.1 to 1.9 dL/g, preferably 0.1 to 1.4 dL/g, and more preferably 0.1 to 1.2 dL/g. Biocompatible polymers with an intrinsic viscosity of less than 0.1 dL/g may decompose too quickly and thus may have difficulty in continuously releasing varenicline for a desired period of time, and biocompatible polymers with an intrinsic viscosity exceeding 0.9 dL/g may decompose slowly and thus release a small amount of varenicline, which may not result in an efficacy.

In the present invention, the content range of the biocompatible polymer included in the microsphere may have a lower limit of 50 wt % or more, 55 wt % or more, 60 wt % or more, 65 wt % or more, or 70 wt % or more, and an upper limit of 98 wt % or less, 96 wt % or less, 94 wt % or less, 92 wt % or less, 90 wt % or less, or 86 wt % or less, based on the total weight of the microsphere. The content range of the biocompatible polymer may be set by a combination of any of the lower limit values and any of the upper limit values.

Specifically, the content range of the biocompatible polymer may be included as 50 to 98 wt %, 55 to 96 wt %, 60 to 94 wt %, 65 to 92 wt %, 70 to 90 wt %, or 70 to 86 wt %.

If the biocompatible polymer contained in the microsphere is included in less than 50 wt %, the distribution of varenicline hemipamoate may relatively increase, which may cause problems such as initial excessive release or inability to maintain efficacy for a desired period of time. If the biocompatible polymer is included in excess of 98 wt %, the amount to be administered to the patient may become too large, making administration difficult or impossible.

As the biocompatible polymer compound, at least one selected from the group consisting of polyglycolic acid, polylactic acid, polyglycolide, polylactide, polylactic-co-glycolic acid, polylactide-co-glycolide (PLGA), polyphosphazine, polyiminocarbonate, polyphosphoester, polyanhydride, polyorthoester, copolymer of lactic acid and caprolactone, polycaprolactone, polyhydroxyvalerate, polyhydroxybutyrate, polyamino acid, and copolymer of lactic acid and amino acid may be used, and most preferably, polylactide-co-glycolide (PLGA) may be used.

In the present invention, the microsphere may contain impurities (components other than varenicline hemipamoate and the biocompatible polymer) contained during the preparing process in an amount of 5 wt % or less based on the total weight of the microsphere. When such impurities are taken into consideration, the content of the biocompatible polymer may be reduced by the amount of the impurities.

The microsphere of the present invention has an excellent actual varenicline loading rate based on a varenicline free base, thereby reducing the dosage and improving the convenience of administration. In addition, since the encapsulation rate of varenicline is excellent, productivity can be improved during mass production.

In one embodiment of the present invention, the microspheres can provide the encapsulated varenicline in a controlled or extended release form. The controlled or extended release form can be understood to have the same meaning as “sustained release”, “controlled release” or “delayed release”.

As one embodiment of the present invention, the microsphere may have a characteristic in which the release of the encapsulated varenicline continues for 30 days or more or 40 days or more in an in vitro environment.

In addition, varenicline contained in the microsphere may have the characteristic of being released within 7 days in an in vitro environment at 25 wt % or less, 24 wt % or less, 23 wt % or less, 22% or less, 20 wt % or less, 18 wt % or less, 16 wt % or less, 14 wt % or less, 12 wt % or less, or 10 wt % or less, and the remainder being continuously released after 7 days.

In addition, varenicline contained in the microsphere may have the characteristic of being released at 60 wt % or less, 50 wt % or less, 45 wt % or less, 40 wt % or less, 20 wt % or less, or 15 wt % or less within 14 days in an in vitro environment, and the remainder being continuously released after 14 days.

In addition, varenicline contained in the microsphere may have the characteristics of being released at 85 wt % or less, 83 wt % or less, 81 wt % or less, 80 wt % or less, 70 wt % or less, 60 wt % or less, or 55 wt % or less within 28 days in an in vitro environment, and the remainder being continuously released after 28 days.

In addition, varenicline contained in the microsphere may have the characteristics of being released at 95 wt % or less, 94 wt % or less, 93 wt % or less, 92 wt % or less, or 90 wt % or less within 42 days in an in vitro environment, and the remainder being continuously released after 42 days.

As one embodiment of the present invention, the microsphere containing varenicline hemipamoate may be prepared by a solvent evaporation or extraction method using an emulsion, more preferably, an O/W (oil-in-water) type solvent evaporation method in which an O/W type emulsion containing a biocompatible polymer, varenicline hemipamoate, and a dispersion solvent is prepared and the O/W type emulsion is coagulated into the microsphere.

In the present invention, when the microsphere containing varenicline hemipamoate is prepared according to a solvent evaporation method or solvent extraction method using an O/W type emulsion among various microsphere preparation methods known in the art (for example, an O/W type, an O/O type, or a W/0/W type solvent evaporation method or solvent extraction method, a microsphere preparation method by spray drying, a microsphere preparation method by phase separation, etc.), the encapsulation rate of varenicline hemipamoate in the microsphere can be significantly improved.

In order to prepare the microsphere by preparing the O/W type emulsion and coagulating it into the polymer microsphere, first, an O/W type emulsion containing a biocompatible polymer, varenicline hemipamoate, and a dispersion solvent is prepared.

The O/W type emulsion can be prepared using a conventional method known in the art, and more specifically, for the preparation of the O/W type emulsion, a dispersion phase including a biocompatible polymer and varenicline hemipamoate can be prepared by adding it to a dispersion solvent. This varenicline hemipamoate-containing polymer microsphere is prepared by coagulating an emulsion into the microsphere by a solvent evaporation method and/or a solvent extraction method, or by coagulation by an ammonolysis or hydrolysis process. In the case of the ammonolysis process, by the addition of ammonia, and in the case of the hydrolysis process, by the addition of an acid or base, a water-insoluble organic solvent that is converted into a water-soluble solvent by the ammonolysis or hydrolysis reaction is additionally included in the preparation of the emulsion.

In the case of the solvent evaporation method, the present invention is not limited thereto, but for example, the method described in U.S. Pat. Nos. 6,471,996, 5,985,309, and 5,271,945, etc., i.e., dispersing or dissolving a drug in an organic solvent phase in which a polymer compound is dissolved, emulsifying the drug in a dispersion medium such as water to prepare an O/W type emulsion, and then diffusing the organic solvent in the emulsion into the dispersion medium and evaporating it through the air/water interface, thereby forming varenicline hemipamoate-containing polymer microspheres.

The solvent extraction method includes a conventional solvent extraction method used in the preparation of a varenicline hemipamoate-containing polymer microsphere, such as effectively extracting the organic solvent in the emulsion droplets using a large amount of solubilizing solvent.

As a method of simultaneously applying the solvent evaporation method and the solvent extraction method, for example, methods described in U.S. Pat. Nos. 4,389,840, 4,530,840, 6,544,559, 6,368,632, and 6,572,894 can be applied.

The coagulation by the above ammonolysis process refers to a method of coagulating a microsphere by adding ammonia to an O/W type emulsion containing a water-insoluble organic solvent to induce ammonolysis, converting the water-insoluble organic solvent into a water-soluble solvent, as described in, for example, Korean Patent No. 918092.

The coagulation by the hydrolysis process refers to a method of coagulating a microsphere by adding a base such as NaOH, LiOH, KOH, or an acid solution such as HCl or H₂SO₄ to an O/W type emulsion containing a water-insoluble organic solvent, thereby inducing hydrolysis, which is a type of hydrolysis reaction of ester, and converting the water-insoluble organic solvent into a water-soluble solvent, as described in, for example, Korean Patent Application Nos. 2009-109809 and 2010-70407.

In addition, the present invention relates to a method of preparing microspheres, comprising the following steps of:
(a) dispersing varenicline hemipamoate and a biocompatible polymer in one or more solvents to prepare a dispersed phase;
(b) adding the produced dispersed phase to a continuous phase and stirring to form a microsphere; and
(c) removing the solvent.

All of the contents described above regarding microspheres can be applied to the method of preparing a microsphere of the present invention. Therefore, any duplicate descriptions are omitted below.

The step (a) is a step of preparing a dispersion phase containing varenicline hemipamoate and a biocompatible polymer. In the step (a), varenicline hemipamoate may be dispersed or dissolved in an amount of 2 to 100 parts by weight, preferably 4 to 82 parts by weight, more preferably 6 to 67 parts by weight, and still more preferably 7 to 54 parts by weight, based on 100 parts by weight of the biocompatible polymer. Specifically, for example, it may be dispersed or dissolved in an amount of 11 to 43 parts by weight.

The solvent used for organic phase preparation is not particularly limited in type, but dimethyl sulfoxide, methylene chloride, etc. can be used.

The step (b) is a step of solidifying the microsphere by dispersing the dispersed phase prepared in the step (a) in an external continuous phase to prepare an emulsion solution (0/W).

In the step (b), a hydrophilic polymer may be included as a surfactant, and the type of the surfactant is not particularly limited, and any surfactant may be used as long as it can help the dispersed phase including varenicline hemipamoate and a biocompatible polymer to form a stable droplet dispersed phase within the external continuous phase.

The hydrophilic polymer may preferably be selected from the group consisting of methylcellulose, polyvinylpyrrolidone, carboxymethylcellulose, lecithin, gelatin, polyvinyl alcohol, polyoxyethylene-polyoxypropylene block copolymer, polyoxyethylene sorbitan fatty acid ester, and polyoxyethylene castor oil derivatives and mixtures thereof, and most preferably, polyvinyl alcohol may be used.

In the step (b), the external continuous phase may be a hydrophilic polymer aqueous solution of 0.1 to 5% (w/v), preferably 0.5 to 3% (w/v), wherein the weight average molecular weight of the hydrophilic polymer may be 10,000 to 30,000, and the degree of hydrolysis may be 80 to 90%.

In the step (b), the dispersed phase containing varenicline hemipamoate and a biocompatible polymer prepared in the step (a) is added to the external continuous phase containing the hydrophilic polymer by drop-by-drop or by using an in-line mixer, and stirred vigorously to prepare an emulsion solution (0/W). In this process, the varenicline hemipamoate is encapsulated into the biocompatible polymer microspheres.

Afterwards, in the step (c), the solvent is removed, and after conventional filtration and washing, the desired microsphere can be obtained. That is, a step of washing the obtained microsphere using an organic solvent such as ethanol may be included to enhance the initial release inhibition effect, if necessary.

As one embodiment of the present invention, the weight of varenicline hemipamoate encapsulated in the microsphere obtained according to the preparation method may be 50 wt % or more, preferably 60 wt % or more, more preferably 70 wt % or more, still more preferably 80 wt % or more, most preferably 90 wt % or more, and particularly preferably 93 wt % or more, based on the weight of varenicline hemipamoate dissolved in the step (a).

In addition, the present invention provides a pharmaceutical composition for the prevention or treatment of diseases caused by cholinergic receptor activation disorder, comprising the microsphere and a pharmaceutically acceptable carrier.

In addition, the present invention provides a method of preventing or treating diseases caused by cholinergic receptor activation disorder by administering an effective amount of the microsphere to a mammal, including a human.

Furthermore, the present invention provides the use of the microsphere for the preparation of a medicament for the prevention or treatment of diseases caused by cholinergic receptor activation disorder.

The diseases caused by cholinergic receptor activation disorder refers to the use for preventing or treating, for example, inflammatory bowel disease, irritable bowel syndrome, spastic dystonia, chronic pain, acute pain, nontropical sprue, cystitis, vasoconstriction, anxiety disorders, panic disorder, depression, bipolar disorder, autism, sleep disorders, jet lag syndrome, amyotrophic lateral sclerosis (ALS), cognitive dysfunction, drug/toxicity-induced cognitive impairment, disease-induced cognitive impairment, hypertension, bulimia, anorexia, obesity, cardiac arrhythmias, gastric hypersecretion, ulcers, pheochromocytoma, progressive supranuclear palsy, chemical dependence and addiction (e.g., dependence or addiction to nicotine or tobacco products, alcohol, benzodiazepines, barbiturates, opioids, or cocaine), headache, migraine, stroke, traumatic brain injury (TBI), obsessive-compulsive disorder (OCD), psychosis, Huntington's chorea, tardive dyskinesia, hyperkinesia, dyslexia, schizophrenia, multi-infarct dementia, age-related cognitive decline, epilepsy including absence seizures (absence epilepsy), attention deficit hyperactivity disorder (ADHD), Tourette syndrome.

The pharmaceutical composition of the present invention can be preferably used for the prevention and treatment of particularly nicotine dependence and addiction (smoking cessation aid therapy) among the above diseases.

The pharmaceutical composition according to the present invention can be formulated for oral administration and can be formulated in various forms, such as a tablet, a film, a suspension, a granule, a gel, a pill, a tincture, a decoction, an infusion, a spirit, a fluidextract, an elixir, an extract, a syrup, a powder, an aromatic water, a lemonade, etc. In addition, the tablet may be formulated in various forms, such as an orally disintegrating tablet, a mucoadhesive tablet, a dispersible tablet, a sublingual tablet, a buccal tablet, a chewable tablet, an effervescent tablet, a solution tablet, etc., but is not limited thereto.

The pharmaceutical composition for oral administration according to the present invention may further comprise a pharmaceutically acceptable carrier which can be typically added to the pharmaceutical composition.

The pharmaceutically acceptable carrier may include additives such as excipients, plasticizers, disintegrants, diluents, solvents, penetration enhancers, preservatives, buffers, gel forming agents, lubricants, carriers, stabilizers, gels, dyes, pigments, surfactants, inert fillers, adhesives, texturizers, softeners, emulsifiers and mixtures thereof commonly used in the pharmaceutical field.

Examples of the excipients include, but are not limited to, cellulose, methylcellulose, ethylcellulose, hydroxypropyl cellulose and hydroxymethylcellulose, polypropylpyrrolidone, polyvinylpyrrolidone, gelatin, gum arabic, polyethylene glycol, starch, natural and synthetic gums (e.g., gum arabic, alginate and gum arabic) and mannitol, microcrystalline cellulose, anhydrous calcium hydrogen phosphate, sorbitol, L-HPC (low-substituted hydroxypropyl cellulose), pregelatinized starch, lactose and/or mixtures thereof.

Examples of the lubricants include, but are not limited to, calcium stearate, glyceryl monostearate, glyceryl palmitostearate, magnesium stearate, polyethylene glycol, sodium benzoate, sodium lauryl sulfate, sodium stearyl fumarate, stearic acid, talc, and zinc stearate.

Examples of the disintegrants include, but are not limited to, sodium starch glycolate, sodium carboxymethylcellulose, calcium carboxymethylcellulose, croscarmellose sodium, polyvinyl pyrrolidone, methyl cellulose, microcrystalline cellulose, powdered cellulose, lower alkyl-substituted hydroxypropyl cellulose, polacrilin potassium, starch, pregelatinized starch, and sodium alginate.

In addition, the formulation for oral administration according to the present invention may additionally contain, if necessary, a sweetener, a flavoring agent, and/or a coloring agent.

The pharmaceutical composition according to the present invention can be formulated for parenteral administration, and can be formulated in the form of, but not limited to, injections, creams, lotions, ointments for external use, oils, moisturizers, gels, aerosols, patches, and nasal inhalers. These formulations are described in a generally known prescription book in all pharmaceutical chemistry (Remington's Pharmaceutical Science, 19th ed., Mack Publishing Company, Easton, PA, 1995).

The pharmaceutical composition for parenteral administration according to the present invention may contain the microspheres alone or may further comprise a pharmaceutically acceptable carrier for parenteral administration which can be conventionally added to the pharmaceutical composition. In addition, it may further contain an excipient or a diluent. The carrier includes all kinds of solvents, dispersion media, oil-in-water or water-in-oil emulsions, aqueous compositions, liposomes, microbeads and microsomes.

The parenteral administration carrier may include water, suitable oils, saline solution, aqueous glucose and glycol, and may additionally include stabilizers and preservatives.

The suitable stabilizers include antioxidants such as sodium bisulfite, sodium sulfite or ascorbic acid. The suitable preservatives include benzalkonium chloride, methyl- or propyl-paraben and chlorobutanol.

The pharmaceutical composition of the present invention may further include, in addition to the components, a lubricant, a wetting agent, a sweetening agent, a flavoring agent, an emulsifier, a suspending agent, etc. Other pharmaceutically acceptable carriers and preparations may be referred to as described in the following literature (Remington's Pharmaceutical Sciences, 19th ed., Mack Publishing Company, Easton, PA, 1995).

The parenteral administration methods of the present invention include, but are not limited to, intravenous, intramuscular, intraarterial, intramedullary, intrathecal, intracardiac, transdermal, subcutaneous, intraperitoneal, intranasal, enteral, intravaginal, intrapulmonary, suppository, topical, sublingual, or rectal administration to a patient (e.g., a person in need of such a drug) or other animals.

In the present invention, ‘treatment’ means any act of improving or beneficially changing disease, disorder and its symptoms (whether acute or chronic) by administering a pharmaceutical composition. In addition, the ‘treatment’ broadly includes the meaning of ‘prevention’, and ‘prevention’ means any act of suppressing or delaying the onset of a disease and its symptoms by administering a pharmaceutical preparation. The ‘treatment’ includes, for example, interference, alleviation, improvement, cessation, suppression, delay and reversal of the progression of a disease, disorder and its symptoms (whether acute or chronic).

A preferred total dosage of the pharmaceutical composition or drug of the present invention may be about 0.01 mg to 2,000 mg, most preferably 0.1 mg to 1,000 mg per day based on varenicline free base. However, the dosage, frequency and duration of administration of the pharmaceutical composition will vary depending on factors such as the nature and severity of the condition to be treated, the age and general health of the subject (host), and the tolerance of the subject (host) to the active ingredient. In consideration of these points, a person having ordinary skill in the art will be able to determine an appropriate effective dosage of the composition of the present invention. The pharmaceutical composition according to the present invention is not particularly limited in its formulation, administration route, and administration method as long as it exhibits the effects of the present invention.

Hereinafter, the present invention will be described in detail based on the following Examples. However, the following Examples are only intended to illustrate the present invention, and the scope of the present invention is not limited thereto.

### Example 1: Preparation of Varenicline Hemipamoate

4.24 g of varenicline (Varenicline base, manufacturer: Lee Pharma) and 3.88 g of pamoic acid (manufacturer: Sigma Aldrich) were added to a mixed solvent of 55 mL of ultrapure water and 11 mL of dimethyl sulfo ide. Next, the mixture was heated to 70° C. and stirred at a speed of 300 times per minute to prepare a solution. The solution was stored at room temperature for 1 hour, and the temperature was slowly lowered to obtain a precipitate. The precipitate (containing varenicline hemipamoate) was washed with 10 mL of water and dried at 60° C. for 8 hours to obtain varenicline hemipamoate.

### Example 2: Preparation of Microsphere Containing Varenicline Hemipamoate

0.25 g of varenicline hemipamoate (Varenicline hemi-pamoate salt) prepared in the above Example 1 was dissolved in 1.1 g of dimethyl sulfoxide (manufacturer: Sigma Aldrich), 1.0 g of biocompatible polymer (B6012-4, manufacturer: Evonik) was dissolved in 5.32 g of methylene chloride (manufacturer: Deoksan), and these were mixed to complete a dispersed phase.

The continuous phase used a 1% (w/v) polyvinyl alcohol (Sigma Aldrich, molecular weight: 13,000-23,000) aqueous solution. 1,000 ml of the continuous phase was placed in a manufacturing tank, maintained at 25° C., and the prepared dispersed phase was injected and stirred with a homogenizer to prepare a microsphere. Thereafter, the organic solvent was removed at 25° C. for 20 hours. The prepared microspheres were washed several times with water for injection, the residual polyvinyl alcohol was removed, and the microsphere was freeze-dried.

### Comparative Example 1: Preparation of Varenicline Pamoate

A varenicline solution was prepared by dissolving 1.00 g of varenicline (Varenicline base, manufacturer: Lee Pharma) in 50 ml of ultrapure water and adding 325 μl of acetic acid. Disodium pamoate 1.938 g (manufacturer: Addtek chemical shanghai co LTD) was completely dissolved in 100 ml of ultrapure water to prepare a disodium pamoate solution. The varenicline solution was placed in a 3-neck flask, stirred at a speed of 300 times per minute, and the disodium pamoate solution was slowly added to obtain varenicline pamoate, which was then freeze-dried.

### Comparative Example 2: Preparation of Microsphere Containing Varenicline Pamoate

0.25 g of varenicline pamoate (Varenicline pamoate salt) prepared in the above Comparative Example 1 was dissolved in 1.1 g of dimethyl sulfoxide (manufacturer: Sigma Aldrich), 1.0 g of biocompatible polymer (B6012-4, manufacturer: Evonik) was dissolved in 5.32 g of methylene chloride (manufacturer: Deoksan), and these were mixed to complete a dispersed phase.

The continuous phase used a 1% (w/v) polyvinyl alcohol (Sigma Aldrich, molecular weight: 13,1000-23,000) aqueous solution. 1,000 ml of the continuous phase was placed in a manufacturing tank, maintained at 25° C., and the prepared dispersed phase was injected and stirred with a homogenizer to prepare a microsphere. Afterwards, the organic solvent was removed at 25° C. for 20 hours. The prepared microsphere was washed several times with water for injection, and the residual polyvinyl alcohol was removed and the microsphere was freeze-dried.

### Comparative Example 3: Preparation of Microsphere Containing Varenicline Tartrate

0.1 g of varenicline tartrate (Varenicline tartrate salt) was dissolved in 3.3 g of dimethyl sulfoxide (manufacturer: Sigma Aldrich), 0.9 g of biocompatible polymer (Resomer 504H, manufacturer: Evonik) was dissolved in 3.99 g of methylene chloride (manufacturer: Deoksan), and these were mixed to complete the dispersion phase.

The continuous phase used a 0.51. (w/v) polyvinyl alcohol (Sigma Aldrich, molecular weight: 13,000-23,000) aqueous solution. 1,000 ml of the continuous phase was placed in a manufacturing tank and maintained at 25° C., and the prepared dispersed phase was injected and stirred with a homogenizer to prepare a microsphere. Afterwards, the organic solvent was removed at 25° C. for 20 hours. The prepared microsphere was washed several times with water for injection, and then the residual polyvinyl alcohol was removed and the microsphere was freeze-dried.

### Test Example 1. Measurement of Microsphere Morphology

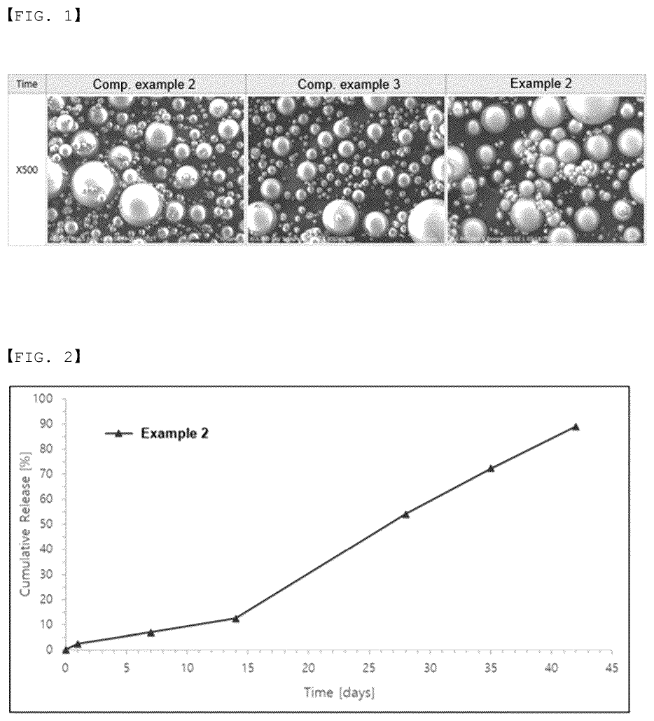

In order to observe the morphology of the microsphere prepared in the above Example 2 and Comparative Example 2 and Comparative Example 3, scanning electron microscopy (SEM) was used. About 20 mg of microsphere was fixed to an aluminum stub and mounted on a SEM (equipment name: Hitachi TM4000 Plus) to observe the surface of the microsphere. All images were observed with a 5 kV electron beam at a magnification of about 500×. The observation results are shown in FIG. 1.

### Test Example 2. Measurement of Varenicline Loading Rate and Encapsulation Rate in Microsphere

Approximately 10 mg of the microspheres prepared in the above Example 2 and Comparative Example 2 were placed in a 20 mL volumetric flask, completely dissolved in 2 mL of acetonitrile (manufacturer: Honeywell), and then adjusted to the mark with 80% methanol (manufacturer: Honeywell) and filtered through a 0.45 μm syringe filter. This solution was detected by ultraviolet-visible spectrophotometry using HPLC (equipment name: Agilent). The column packing was L1, the internal diameter was 4.6 mm×150 mm, and the thickness was 5 μm. The results of the confirmation are shown in Table 1 below.

TABLE 1

| | | Varenicline | |
|---|---|---|---|
| | | free base | Encapsulation |
| | Drug used | loading rate (wt %) | rate(wt %) |
| Example 2 | Varenicline | 9.73 | 93.40 |
| | hemipamoate | | |
| Comparative | Varenicline pamoate | 6.40 | 90.90 |
| Example 2 | | | |
| Comparative | Varenicline tartrate | 1.3 | 22 |
| Example 3 | | | |

From the results in Table 1 above, it can be confirmed that the varenicline actual loading rate of the microsphere of Example 2 of the present invention is significantly superior to the microspheres of Comparative Example 2 and Comparative Example 3.

### Test Example 3: Evaluation of In Vitro Release Test and Initial Release Rate

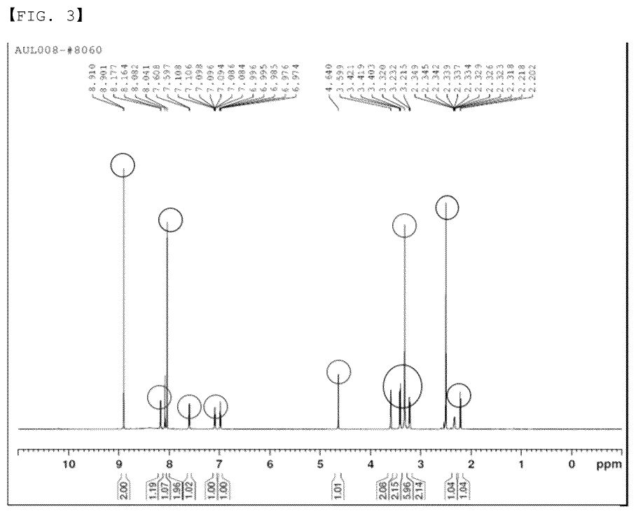

Approximately 20 mg of the microspheres prepared in the above Example 2 and Comparative Example 2 were each placed in an 8 mL amber vial, 8 mL of 0.1% poloxamer in pH 7.4 PBS solution was added, and the mixture was stirred at 100 rpm and maintained at 37° C. In order to measure the release amount over a certain period of time, 2 mL of the supernatant was taken after centrifugation and filtered through a 0.22 μm RC filter. The solution was placed in a vial and detected by a UT-visible spectrophotometer using HPLC (equipment name: Agilent 1260). The column packing was L1, the internal diameter was 4.6 mm×150 mm, and the thickness was 5 μm. The results of the experiment are shown graphically in FIG. 2.

From FIG. 2, it can be confirmed that the microsphere prepared in the above Example 2 exhibits an appropriate initial burst of drug release with 2 wt % of the drug released over 1 day, and that the drug is continuously released consistently for more than 40 days.

### Test Example 4: Analysis of Varenicline Hemipamoate Crystal

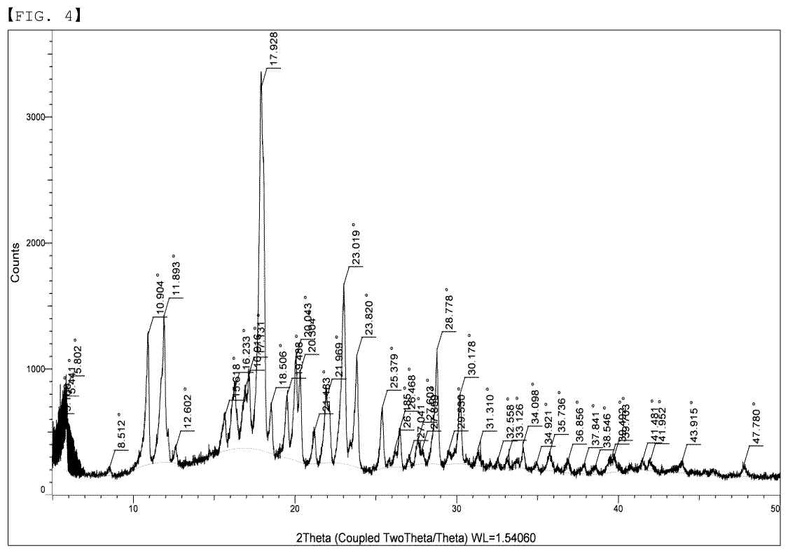

The varenicline hemipamoate crystals prepared in the above Example 1 were analyzed using NMR, XRD, and thermogravimetric analysis (TGA/DSC), and the results are shown in FIGS. 3 to 5.

Additionally, for comparison, varenicline pamoate crystals prepared in Comparative Example 1 were analyzed using NMR, and the results are shown in FIG. 6.

From the above FIGS. 3 to 5, it can be confirmed that the compound obtained in the above Example 1 is a varenicline hemipamoate crystal.

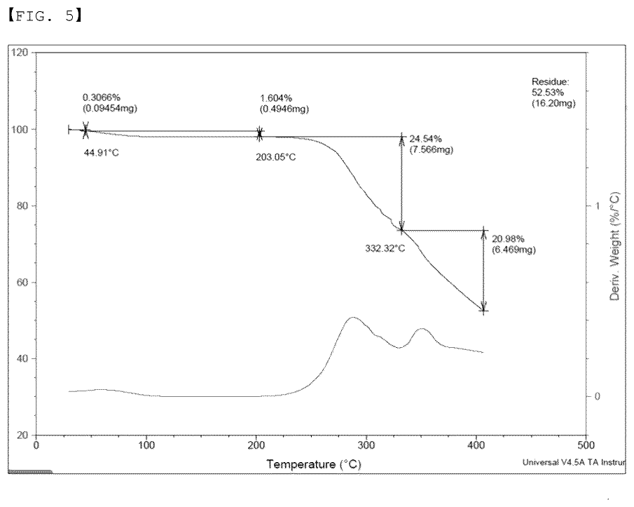

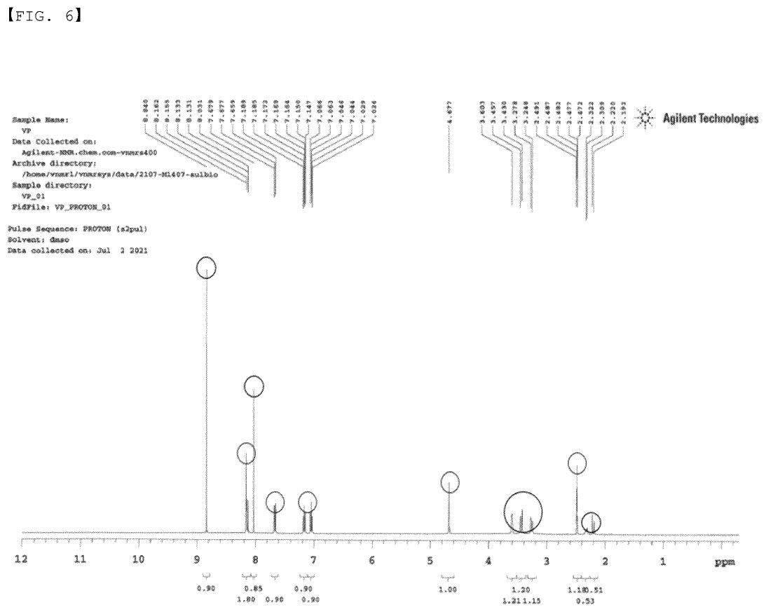

That is, since the peaks of varenicline and pamoic acid are confirmed in the above FIG. 3, and multiple peaks are confirmed in FIG. 4, it can be seen that the compound prepared in Example 1 is a varenicline hemipamoate crystal with crystallinity. In addition, since thermal decomposition occurs at 203.5° C. in FIG. 5, it can be confirmed from this data that the compound prepared in Example 1 is a varenicline hemipamoate crystal.

1. A microsphere comprising varenicline hemipamoate and a biocompatible polymer.

2. The microsphere of claim 1, wherein the varenicline hemipamoate is contained in an amount of 2 to 50 wt % based on the total weight of the microsphere.

3. The microsphere of claim 1, wherein the varenicline hemipamoate is contained in an amount of 10 to 30 wt % based on the total weight of the microsphere.

4. The microsphere of claim 2, wherein the biocompatible polymer is contained in an amount of 50 to 98 wt % based on the total weight of the microsphere.

5. The microsphere of claim 1, wherein the biocompatible polymer is at least one selected from polylactic acid, polylactide, polylactic-co-glycolic acid, polylactide-co-glycolide (PLGA), polyphosphazine, polyiminocarbonate, polyphosphoester, polyanhydride, polyorthoester, copolymer of lactic acid and caprolactone, polycaprolactone, polyhydroxyvalerate, polyhydroxybutyrate, polyamino acid, and copolymer of lactic acid and amino acid.

6. The microsphere of claim 1, which exhibits a sustained release of varenicline hemipamoate for more than 30 days.

7. The microsphere of claim 1, which exhibits a sustained release of varenicline hemipamoate for more than 40 days.

8. The microsphere of claim 1, wherein the amount of varenicline hemipamoate released within 7 days contained in the microsphere is 25 wt % or less.

9. The microsphere of claim 1, wherein the amount of varenicline hemipamoate released within 14 days contained in the microsphere is 60 wt % or less.

10. The microsphere of claim 1, wherein the microsphere is prepared by an O/W (oil-in-water) type solvent evaporation method or solvent extraction method containing a biocompatible polymer, varenicline hemipamoate, and a dispersion solvent.

11. A method of preparing a microsphere, comprising the following steps of:
(a) dispersing varenicline hemipamoate and a biocompatible polymer in one or more solvents to prepare a dispersed phase;
(b) adding the prepared dispersed phase to a continuous phase and stirring to form a microsphere; and
(c) removing the solvent.

12. The method of preparing a microsphere of claim 11, wherein the weight of varenicline hemipamoate encapsulated in the microsphere obtained according to the preparation method is 50 wt % or more based on the weight of varenicline hemipamoate dissolved in the step (a).

13. The method of preparing a microsphere of claim 11, wherein the weight of varenicline hemipamoate encapsulated in the microsphere obtained according to the preparation method is 90 wt % or more based on the weight of varenicline hemipamoate dissolved in the step (a).

14. The method of preparing a microsphere of claim 11, wherein the biocompatible polymer is at least one selected from polylactic acid, polylactide, polylactic-co-glycolic acid, polylactide-co-glycolide (PLGA), polyphosphazine, polyiminocarbonate, polyphosphoester, polyanhydride, polyorthoester, copolymer of lactic acid and caprolactone, polycaprolactone, polyhydroxyvalerate, polyhydroxybutyrate, polyamino acid, and copolymer of lactic acid and amino acid.

15. A pharmaceutical composition for the prevention or treatment of diseases caused by cholinergic receptor activation disorder, comprising the microsphere according to claim 1 and a pharmaceutically acceptable carrier.

16. The pharmaceutical composition of claim 15, wherein the diseases caused by cholinergic receptor activation disorder include inflammatory bowel disease, irritable bowel syndrome, spastic dystonia, chronic pain, acute pain, nontropical sprue, cystitis, vasoconstriction, anxiety disorders, panic disorders, depression, bipolar disorder, autism, sleep disorders, jet lag syndrome, amyotrophic lateral sclerosis (ALS), cognitive dysfunction, drug/toxicity-induced cognitive impairment, disease-induced cognitive impairment, hypertension, bulimia, anorexia, obesity, cardiac arrhythmias, gastric hypersecretion, ulcers, pheochromocytoma, progressive supranuclear palsy, chemical dependence and intoxication, headache, migraine, stroke, traumatic brain injury (TBI), obsessive-compulsive disorder (OCD), psychosis, Huntington's chorea, tardive dyskinesia, hyperkinesia, dyslexia, schizophrenia, multi-infarct dementia, age-related cognitive decline, epilepsy including absence seizures (absence epilepsy), attention-deficit hyperactivity disorder (ADHD), or Tourette syndrome.

17. The pharmaceutical composition of claim 15, wherein the diseases caused by cholinergic receptor activation disorder are nicotine dependence and addiction.

18. A method for preventing or treating a disease caused by cholinergic receptor activation disorder in a subject in need thereof, comprising administering a composition comprising the microsphere according to claim 1 to the subject.

19. The method of claim 18, wherein the disease caused by cholinergic receptor activation disorder is inflammatory bowel disease, irritable bowel syndrome, spastic dystonia, chronic pain, acute pain, nontropical sprue, cystitis, vasoconstriction, anxiety disorders, panic disorders, depression, bipolar disorder, autism, sleep disorders, jet lag syndrome, amyotrophic lateral sclerosis (ALS), cognitive dysfunction, drug/toxicity-induced cognitive impairment, disease-induced cognitive impairment, hypertension, bulimia, anorexia, obesity, cardiac arrhythmias, gastric hypersecretion, ulcers, pheochromocytoma, progressive supranuclear palsy, chemical dependence and intoxication, headache, migraine, stroke, traumatic brain injury (TBI), obsessive-compulsive disorder (OCD), psychosis, Huntington's chorea, tardive dyskinesia, hyperkinesia, dyslexia, schizophrenia, multi-infarct dementia, age-related cognitive decline, epilepsy including absence seizures (absence epilepsy), attention-deficit hyperactivity disorder (ADHD), or Tourette syndrome.

20. The method of claim 18, wherein the disease caused by cholinergic receptor activation disorder is nicotine dependence or nicotine addiction.
